SEQUENCE LISTING (2 sequences)

SEQ ID NO: 1 - DNA, 20 bp, synthetic construct
  source 1..20: mol_type = other DNA; note = nucleotide sequence for 16S rRNA primer 27F
agagtttgatcctggctcag

SEQ ID NO: 2 - DNA, 19 bp, synthetic construct
  source 1..19: mol_type = other DNA; note = nucleotide sequence for 16S rRNA universal primer 1492R
ggttaccttgttacgactt

(19) United States
(12) Patent Application Publication
(10) Pub. No.: US 2025/0320454 A1
(43) Pub. Date: Oct. 16, 2025

(54) ACIDAMINOCOCCUS FERMENTANS STRAIN AND USE THEREOF IN IMPROVING RUMEN FERMENTATION

(71) Applicant: Institute of Feed Research of CAAS, Beijing (CN)

(72) Inventors: Yan TU, Beijing (CN); Shengyang XU, Beijing (CN); Xiaoran FENG, Beijing (CN); Junnan MA, Beijing (CN); Chuntao YANG, Beijing (CN)

(21) Appl. No.: 19/078,992

(22) Filed: Mar. 13, 2025

(30) Foreign Application Priority Data
Apr. 11, 2024 (CN) 202410433548.9

(51) Int. Cl. C12N 1/20; A23K 10/18

(52) U.S. Cl. CPC C12N 1/205; A23K 10/18

(57) ABSTRACT

An Acidaminococcus fermentans strain and use thereof in improving rumen fermentation are provided, and relates to the technical field of microorganisms. An acid-producing strain of Acidaminococcus fermentans P47 is obtained by screening, which has desirable passage stability and safety. The strain can increase a ratio of butyric acid in the rumen of a ruminant, improve rumen fermentation parameters of a pre-weaning calf, promote rumen fermentation, improve a rumen environment, and increase an average daily gain (ADG) of the ruminant.

### CROSS REFERENCE TO RELATED APPLICATION

This patent application claims the benefit and priority of Chinese Patent Application No. 2024104335489 entitled “ACIDAMINOCOCCUS FERMENTANS Strain and Use Thereof in Improving Rumen Fermentation” filed with the China National Intellectual Property Administration on Apr. 11, 2024, the disclosure of which is incorporated by reference herein in its entirety as part of the present application.

### REFERENCE TO SEQUENCE LISTING

A computer readable XML file entitled “GWP20241208018-Sequence Listing”, that was created on Mar. 3, 2025, with a file size of about 3,319 bytes, contains the sequence listing for this application, has been filed with this application, and is hereby incorporated by reference in its entirety.

### TECHNICAL FIELD

An Acidaminococcus fermentans strain and use thereof in improving rumen fermentation are provided, and relates to the technical field of microorganisms.

### BACKGROUND

Calves are the reserve of dairy cows. The nursing quality of pre-weaning calves is crucial to their production performance in adulthood. The growth rate during lactation is correlated with the milk production in the first lactation period. Every 100 g increase in average daily gain (ADG) of pre-weaning calves can lead to an 85 kg to 111 kg increase in milk production in the first lactation period of dairy cows. Moreover, the ADG of pre-weaning calves is related to the reproductive performance of cows. Female pre-weaning calves with higher ADG have earlier first estrus, earlier age of artificial insemination, and earlier calving, thus improving a production efficiency of the dairy industry.

The rumen, as a unique digestive organ of ruminants, directly affects the growth and development of ruminants. Fermentation in the rumen driven by rumen microbes provides approximately 70% of the volatile fatty acids (VFAs) for host growth and development. Rumen microbes function as the key driving force for rumen fermentation, which can affect rumen fermentation, thereby changing the host phenotypes, such as ADG. Studies have shown that 25.3% of the changes in ADG of heifers can be interpreted by the rumen microbes. Similarly, some studies have found that the enrichment of acid-producing bacteria in the rumen would lead to an increase in the concentration of total VFAs and butyric acid in the rumen, thereby increasing the ADG of female pre-weaning Holstein calves.

At present, most domestic and foreign studies only focus on the exploration of the relationship between rumen microbes and the ADG of pre-weaning calves based on sequencing, but seldom key rumen microbes have been isolated by studies in vitro.

### SUMMARY

In the present disclosure, an acid-producing strain of Acidaminococcus fermentans is discovered after screening and testing a large number of strains, which is capable of solving the lack of rumen microbes that promote the ADG of pre-weaning calves.

The present disclosure provides an Acidaminococcus fermentans strain, which is designated Acidaminococcus fermentans P47 with a deposit number of CGMCC No. 40799.

In the present disclosure, the Acidaminococcus fermentans P47 is screened from the contents in the rumen of female pre-weaning Holstein calves. The strain was deposited in the China General Microbiological Culture Collection Center (CGMCC) on Dec. 27, 2023, with a taxonomic designation of Acidaminococcus fermentans and a deposit number of CGMCC No. 40799; and the CGMCC is located at Institute of Microbiology, Chinese Academy of Sciences, Building No. 3, No. 1 West Beichen Road, Chaoyang District, Beijing 100101, China.

A method for isolating and identifying the Acidaminococcus fermentans P47 includes the following steps:
step a, finding the strain;
step b, isolating and purifying the strain through a bacterial isolation test; and
step c, identifying the strain through morphological characteristics, physiological and biochemical characteristics, and 16sRNA gene sequence.

In some embodiments, the strain finding in step a includes the following steps:
S1, selecting 8 healthy female Holstein calves with high ADG and 8 healthy female Holstein calves with low ADG that were 60-day-old, collecting their rumen contents and placing in tubes for cryopreservation in liquid nitrogen, followed by conducting metagenomics sequencing analysis; and
S2, finding an Acidaminococcus fermentans strain enriched in rumen of the calves with high ADG using linear discriminant analysis.

In some embodiments, the bacterial isolation test in step b includes the following steps:
S1, selecting 3 healthy female 60-day-old Holstein calves with high ADG, collecting their rumen contents, mixing uniformly, putting in a thermos flask filled with carbon dioxide, and quickly bringing it back to the laboratory;
S2, diluting fresh rumen contents 10³, 10⁴, and 10⁵ times with sterile PBS and coating on a plate, and culturing in a solid Peptone Yeast Glucose (PYG) medium under anaerobic conditions at 37° C. for 48 h;
S3, after colonies grow out, selecting obvious single colonies for purification by streaking on a PYG solid medium 3 times; and
S4, after colony morphologies on the solid medium are consistent, selecting single colonies and culturing in a PYG liquid medium for 24 h and preserving the strain.

Further, the present disclosure provides a microbial inoculant, including the Acidaminococcus fermentans P47 or a fermentation broth thereof.

In some embodiments, the microbial inoculant is a freeze-dried powder of the fermentation broth of the Acidaminococcus fermentans P47.

In some embodiments, the freeze-dried powder of the fermentation broth further includes a cryoprotectant.

In some embodiments, the cryoprotectant is at least one selected from the group consisting of a skimmed milk powder, dextrin, lactose, and sucrose.

Compared with the prior art, the present disclosure has the following beneficial effects:

An acid-producing strain of Acidaminococcus fermentans P47 is obtained by screening, which has desirable passage stability and safety. The strain can increase a ratio of butyric acid in the rumen of a ruminant, improve rumen fermentation parameters of a pre-weaning calf, promote rumen fermentation, improve a rumen environment, and increase an ADG of the ruminant.

### BRIEF DESCRIPTION OF THE DRAWINGS

FIG. 1 shows a growth curve of the Acidaminococcus fermentans P47;

FIG. 2 is a diagram showing acid and alkali resistance of the Acidaminococcus fermentans P47;

FIG. 3 is a diagram showing bile resistance of the Acidaminococcus fermentans P47;

FIG. 4 is a diagram showing artificial gastric fluid resistance of the Acidaminococcus fermentans P47;

FIG. 5 is a diagram showing artificial intestinal fluid resistance of the Acidaminococcus fermentans P47;

FIG. 6 is a diagram showing an acid production performance of the Acidaminococcus fermentans P47; and

FIGS. 7A-7C are diagrams showing influences of the Acidaminococcus fermentans P47 on rumen fermentation.

### DETAILED DESCRIPTION OF THE EMBODIMENTS

In order to make the objectives, technical solutions, and advantages of the examples of the present disclosure clearer, the technical solutions in the present disclosure are described clearly and completely below. Apparently, the described examples are some rather than all of the examples of the present disclosure. All other examples obtained by those of ordinary skill in the art based on the examples of the present disclosure without creative efforts shall fall within the protection scope of the present disclosure.

The techniques or conditions not specified in the examples are all conventional methods or conducted according to the techniques or conditions described in the literature in the field, or according to the product instructions. Reagents or instruments not specified with manufacturers are all conventional products that can be purchased through regular ways.

Milk replacer powder was purchased from Beijing Yuanheng Huizhong Biotechnology Co., Ltd., with a product name of Eurolac Blue, product number: 116601.

The starter was purchased from Zhongbote (Zhangjiakou) Biotechnology Co., Ltd., with a product name of Zhongbote, product number: 06.

### Example 1 Finding the Target Strain

Rumen contents were collected from 8 high-ADG and 8 low-ADG 60-day-old female Holstein calves having no difference in diet composition and total dry matter intake. The rumen contents were stored in liquid nitrogen and subsequently subjected to metagenomics sequencing and VFAs testing. By linear discriminant analysis, it was found that Acidaminococcus fermentans was enriched in the rumen of high-ADG calves, and the ratio of butyric acid in the rumen of high-ADG calves was also significantly higher than that in the low-ADG calves. Spearman correlation analysis revealed that Acidaminococcus fermentans increased the ratio of butyric acid in the rumen of calves, thereby increasing the ADG of calves.

### Example 2 Isolation and Purification of Acidaminococcus fermentans

Rumen content samples were collected from 3 female pre-weaning Holstein calves, and the samples were mixed uniformly and placed in a thermos flask filled with carbon dioxide at 39° C.

All operations were conducted in an anaerobic incubator. 1 mL of the rumen content sample was taken into a test tube, into which 9 mL of sterile PBS solution was added, and the mixture was thoroughly shaken with a vortex shaker to obtain a sample bacterial suspension. Subsequently, gradient dilutions were conducted by diluting 10³, 10⁴, and 10⁵ times, respectively. 0.1 mL of the dilution solution was taken from the sample bacterial suspension diluted 10³, 10⁴, and 10⁵ times, respectively, and coated on a PYG solid plate, and cultured anaerobically at 37° C. for 48 h to 72 h. Single colonies on the medium plate were selected for four-zone streaking, and cultured anaerobically at 37° C. for 24 h to 48 h. The streaking was repeated 3 times to obtain a purified strain.

### Example 3 Identification of Acidaminococcus fermentans

DNA was extracted from the isolated bacterial colonies using a kit purchased from TIANGEN Biotech (Beijing) Co., Ltd. 16SrRNA universal primers (27F: 5′-AGAGTTTGATCCTGGCTCAG-3′ (SEQ ID NO: 1); 1492R: 5′-GGTTACCTTGTTACGACTT-3′ (SEQ ID NO: 2)) were used for PCR, and a reaction system of 30 μL for PCR specifically included: 6 μL of DNA template, 1.2 μL of each primer, 15 μL of Premix Taq™, and 6.6 μL of sterile enzyme-free water. The PCR program included: 94° C., 5 min; and 94° C., 30 S; 56° C., 30 S; 72° C., 45 S for 35 cycles in total; and 72° C., 10 min; the reaction was terminated at 4° C. and PCR products were temporarily stored. The PCR products were sent to Beijing RuiBiotech Co., Ltd. for sequencing. The sequencing results were aligned with the NCBI GenBank. The identification result was Acidaminococcus fermentans, which was designated Acidaminococcus fermentans P47.

### Example 4 Identification of Physicochemical Properties of Acidaminococcus fermentans P47

### 1. Growth Curve

The isolated and purified strain above was activated in a solid medium and inoculated into a PYG liquid medium to allow overnight culture for 12 h. The obtained culture solution was re-inoculated into the liquid medium at a 2% (w/v) inoculation amount, mixed uniformly and placed in a 37° C. anaerobic incubator for culture. The growth curve of the strain was tested and plotted by turbidimetry, where 200 μL of the bacterial solution was taken for absorbance measurement at OD_{600 nm} every 2 h for 24 h.

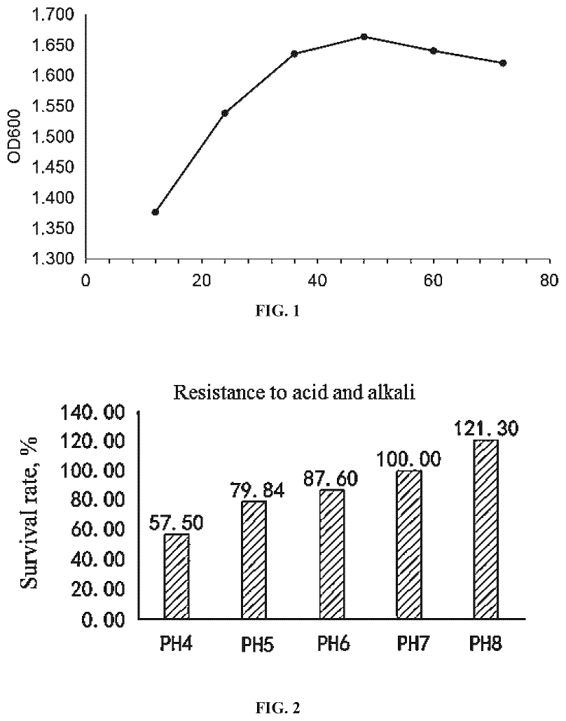

The results were shown in FIG. 1.

### 2. Resistance to Acid and Alkali

The PYG liquid medium was adjusted to pH values of 4, 5, 6, 7 and 8, respectively, and subjected to autoclaving for later use. The bacterial solution cultured overnight for 12 h was inoculated into the PYG liquid medium at each pH value at an inoculation amount of 10% (w/v), with the medium at pH=7 as a control. The culture was conducted in an anaerobic incubator at 37° C. for 24 h, and 200 μL of the bacterial solution from each tube was taken to determine its OD_{600 nm}, and a survival rate of the strain at different pH values was calculated. The formula was: survival rate=(OD₆₀₀ of experimental group÷OD₆₀₀ of control group)×100%. The result shown in FIG. 2 indicates that the Acidaminococcus fermentans P47 had certain resistance to acid and alkali.

### 3. Resistance to Bile Salt

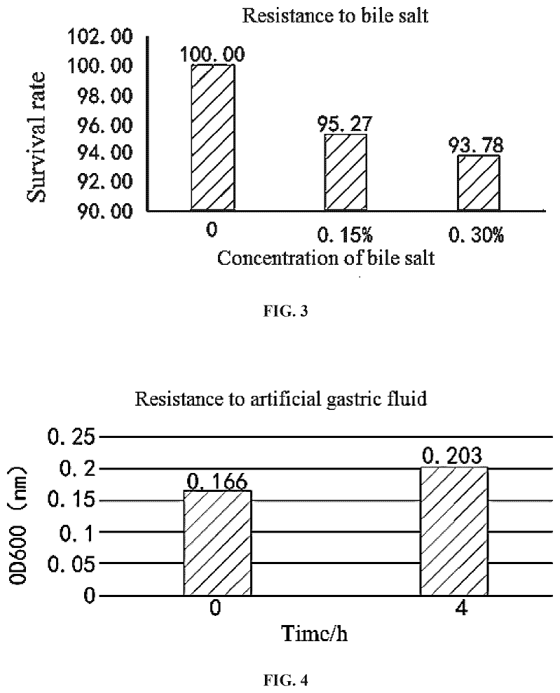

PYG liquid media with bile salt concentrations of 0%, 0.15%, and 0.3% were prepared, and subjected to autoclaving and set aside. The bacterial solution cultured overnight for 12 h was inoculated into PYG liquid medium with different bile salt concentrations at an inoculation amount of 10% (w/v), with the medium without bile salt as a control. The culture was conducted in a shaker at 37° C. for 10 h, and the OD_{600 nm} of each tube of bacterial solution was measured to calculate a survival rate of the strain under 2 bile salt concentrations. The formula was: survival rate=(OD₆₀₀ of experimental group÷OD₆₀₀ of control group)×100%. The result shown in FIG. 3 indicates that the Acidaminococcus fermentans P47 had a certain bile salt resistance.

### 4. Resistance to Artificial Gastric Fluid and Artificial Intestinal Fluid

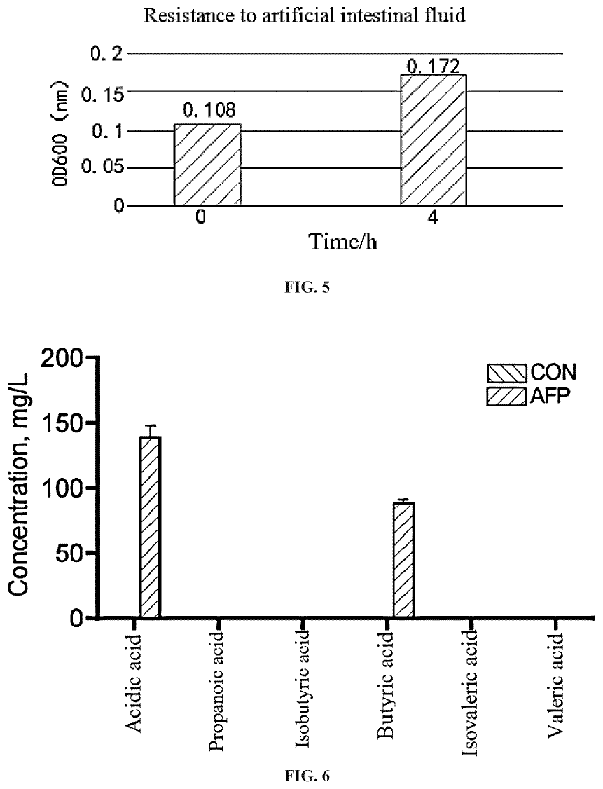

The overnight cultured bacterial solution was washed 2 times with PBS, and the bacterial cells were placed in artificial gastric fluid (PYG liquid medium with pH=3, autoclaved, cooled to room temperature, and into which 1 mg/mL pepsin was aseptically added in an ultra-clean bench) and artificial intestinal fluid (PYG liquid medium with pH=8, autoclaved, cooled to room temperature, and 1 mg/mL trypsin (1:250) was aseptically added in an ultra-clean bench). The bacterial solution was adjusted to a concentration of 10⁸ CFU/mL and cultured in a 37° C. anaerobic incubator, and absorbance at OD_{600 nm} of each bacterial solution was measured at 0 h and 4 h, respectively. The results shown in FIG. 4 and FIG. 5 indicate that the Acidaminococcus fermentans P47 grew to a certain extent in the artificial gastric fluid and artificial intestinal fluid and had a certain resistance.

### 5. Acid Production Performance

The above purified strain was inoculated into PYG liquid medium and cultured anaerobically at 37° C. for 24 h. 0.5 mL of the bacterial solution was taken and added with 0.2 mL of ZnSO₄ and 0.1 mL of K₄Fe(CN)₆ solutions, shaken uniformly, diluted with water to 5 mL, shaken uniformly, centrifuged at 10,000 r/min for 10 min, and 1 mL of a filtrate was taken after filtering through the membrane, diluted 5 times (to 5 mL) with 4 mL of water, and an acid production performance was detected by high-performance liquid chromatography (HPLC). As shown in FIG. 6, the Acidaminococcus fermentans P47 could produce acetic acid and butyric acid, with an acetic acid yield of 149.11 mg/mL and a butyric acid yield of 89.45 mg/mL.

### Example 5 A Microbial Inoculant

This example provided a microbial inoculant, including the Acidaminococcus fermentans P47 that has been deposited in the form of a fermentation broth or a freeze-dried powder, where the fermentation broth contained 1×10⁸ CFU/mL of the live Acidaminococcus fermentans P47; and the freeze-dried powder contained 1×10⁸ CFU/g of the live Acidaminococcus fermentans P47.

### Example 6 In Vitro Fermentation Test

4 healthy Holstein fistulated cows were selected as rumen fluid donors, and rumen fluid was collected after milking and before morning feeding. The rumen fluid was filtered through four layers of gauze and mixed with a Menke medium at a ratio of 1:2 to prepare an artificial rumen fluid, and into which 3 different substrates were added. Each substrate was added into PYG liquid medium without inoculation as a control group; while PYG liquid medium inoculated with the Acidaminococcus fermentans P47 (with an inoculation amount of 1×10⁸ CFU/mL) was added as a treatment group. In vitro fermentation experiments were conducted, where 10 g of substrate, 200 mL of Menke medium, and 100 mL of rumen fluid were added into each fermentation flask, and the flask was filled with carbon dioxide finally and fermented at 39° C. for 48 h.

The experimental design was shown in Table 1.

TABLE 1

| Item | Substrate | Additive |
|---|---|---|
| Control | Milk replacer | PYG liquid medium without inoculation |
| (CON) | Milk replacer + | PYG liquid medium without inoculation |
| | starter | |
| | Starter | PYG liquid medium without inoculation |
| Treatment | Milk replacer | PYG liquid medium inoculated with |
| (AFP) | | Acidaminococcus fermentans |
| | Milk replacer + | PYG liquid medium inoculated with |
| | starter | Acidaminococcus fermentans |
| | Starter | PYG liquid medium inoculated with |
| | | Acidaminococcus fermentans |

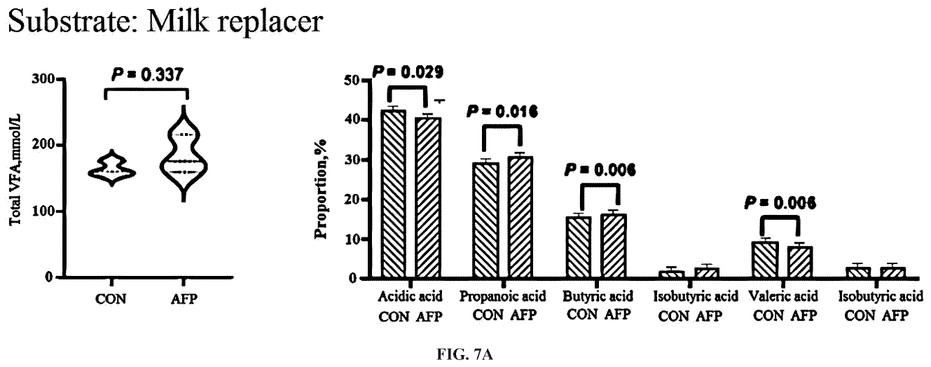

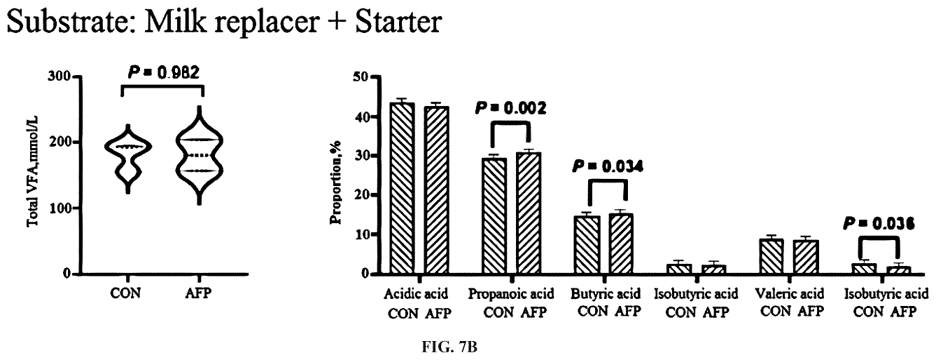

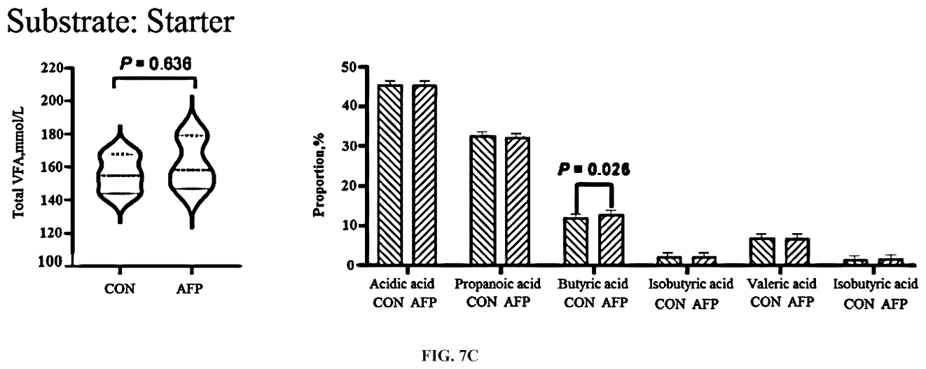

The results shown in FIG. 7 indicate that the addition of the Acidaminococcus fermentans P47 could increase the ratio of butyric acid in rumen fluid (p<0.05) regardless of the substrate.

Finally, it should be noted that the foregoing embodiments are only used to illustrate the technical solutions of the present disclosure, and are not intended to limit the present disclosure. Although the present disclosure is described in detail with reference to the foregoing embodiments, those of ordinary skill in the art should understand that they can still modify the technical solutions described in the foregoing embodiments, or make equivalent substitutions to some technical features therein. These modifications or substitutions do not make the essence of the corresponding technical solutions depart from the spirit and scope of the technical solutions in the embodiments of the present disclosure.

What is claimed is:

1. An Acidaminococcus fermentans strain, wherein the strain is Acidaminococcus fermentans P47 with a deposit number of CGMCC No. 40799.

2. A microbial inoculant, comprising the Acidaminococcus fermentans P47 according to claim 1.

3. The microbial inoculant according to claim 2, wherein the microbial inoculant is a freeze-dried powder of a fermentation broth containing the Acidaminococcus fermentans P47.

4. The microbial inoculant according to claim 3, wherein the freeze-dried powder of the fermentation broth further comprises a cryoprotectant.
